(19) United States
(12) Patent Application Publication
(10) Pub. No.: US 2023/0240541 A1
(43) Pub. Date: Aug. 3, 2023

(54) Ear-Based Core Body Temperature Monitoring System

(71) Applicants: National University of Singapore, Singapore (SR); Well Being Digital Limited, Shatin, N.T., Hong Kong (CN)

(72) Inventors: Cherh Chiet Ivan Low, Singapore (SG); Ming Yip Wallace Wong, Shatin, N.T., Hong Kong (CN)

(21) Appl. No.: 18/003,607

(22) PCT Filed: Jul. 2, 2021
(86) PCT No.: PCT/SG2021/050387

§ 371 Date: Dec. 28, 2022

(30) Foreign Application Priority Data
Jul. 3, 2020 (SG) 10202006454P

(51) Int. Cl. A61B 5/01; A61B 5/0205; A61B 5/00

(52) U.S. Cl. CPC A61B 5/01; A61B 5/02055; A61B 5/6817; A61B 5/7282; A61B 5/683; A61B 5/7267; A61B 5/7275; A61B 5/02438

(57) ABSTRACT

A non-invasive method and system for monitoring core body temperature (Tc) of a user continuously so as to prevent the risk of over-heating. The system comprises a detection unit to be worn in the user's ear for measuring physiological data of the user by a plurality of sensors and an analysis unit connected to the detection unit via a communication link for computing Tc of the user with a prediction model using the physiological data measured by the detection unit where the effects of heart rate and external environmental temperature on auditory canal temperature of the user are taken into account. The sensors comprise two sensors (207, 208) for measuring auditory canal temperatures and sensors (209, 210) for measuring heart rate and external auricle temperature respectively. The prediction model is preferably a random forest prediction model or a linear or polynomial regression model. An over-heating state of the user is determined when the computed Tc is above a threshold level (e.g. above 40° C.).

### FIELD OF THE INVENTION

This invention relates to a system and method for monitoring core body temperature (Tc) of a user continuously. More particularly, this invention relates to a non-invasive method for monitoring core body temperature (Tc) of a user continuously to prevent the risk of over-heating.

### BACKGROUND OF THE INVENTION

Physical exertion in hot and/or humid environments while donning personal protective equipment elevates physiological strain on the body. This is a concern for workers in numerous heat-exposed industries, including but not limited to military personnel, firefighters and mining workers. Day to day tasks in such occupations often entail substantial physical workloads, hot ambient working conditions and may require wearing thick personal protective equipment, thus augmenting thermal work strain. In turn, this could elevate their risk of developing conditions such as exercise associated muscle cramps, heat exhaustion or exertional heat stroke. The latter, classified by extreme hyperthermia (core body temperature exceeding 40° C.) and central nervous system dysfunction, can lead to multiple organ system failure and even death in severe cases. Despite extensive documentation on the prevention and treatment of exertional heat stroke, its prevalence in these industries continues to grow. This suggests that current practices to manage thermal work strain remain inadequate to fully tackle the problem at hand.

Existing heat strain management strategies centre on the identification of high-risk environments and behavioural modifications based on perceived heat stress. However, these strategies fail to consider crucial predisposing factors such as individual differences in metabolic heat production, physical fitness, heat acclimatization/acclimation status and heat injury history. The implementation of personalised physiological monitoring, using wearable technology, is thus proposed as a potential solution to account for individual thermal work strain. The assessment of thermal work strain involves the measurement of several physiological parameters, such as core body temperature, skin temperature, heart rate, and sweat rate.

Yet, there are currently no accurate and practical methods for monitoring of core body temperature (Tc) in occupational settings. At present, available devices for continuous monitoring of Tc are invasive in nature and come at a high cost, for examples rectal probes, oesophageal probes and ingestible telemetric pills. Furthermore, the insertion of either rectal or oesophageal thermistors can cause significant user discomfort and are thus not feasible for implementation on a daily basis. However, despite an improved user comfort when utilising ingestible telemetric pills, this strategy comes with a high cost (e.g. $120 per single-use pill) and is complex to implement due to the need to account for individual differences in gastrointestinal motility. While non-invasive surrogates such as measurement of oral and axilla temperature have been implemented for recording of Tc in clinical settings, these strategies remain unsuitable for use during physical activity due to a high susceptibility to environmental factors and inability to provide continuous Tc measurement.

The viability of the ear as a surrogate measurement site for human Tc has been studied. Tympanic membrane temperature (T_{ty}) was proposed due to the vascularisation of the tympanic membrane by the internal carotid artery which also irrigates the hypothalamus. T_{ty} can be measured by direct contact with the tympanic membrane or indirect measurement of heat emitted from the tympanic membrane and auditory canal. While the former has acceptable correlation with Tc, it is unsafe for use in thermal work strain monitoring as shifting of the thermistor during physical movement can lead to tympanic membrane injury or cause pain should the sensor contact the richly innervated portion of the auditory canal. Indirect measurement of T_{ty} using infrared sensors provides better comfort and safety. However, as a line of sight to the tympanic membrane is necessary for accurate reflection of Tc, factors such as auditory canal shape and/or inadequate depth of insertion can lead to discrepancies. Environmental influences due to poor insulation, sweat condensation and heating of the infrared sensor can also affect measurements.

Monitoring of auditory canal temperature (T_{ac}) is a promising alternative. T_{ac} measurements displayed the best correlation with rectal temperature when the sensor is placed close to the tympanic membrane. Also, no user discomfort as a result of the sensor placement. However, despite its promise, the development of an algorithm based on T_{ac} inputs alone does have its limitations. Specifically, T_{ac} is sensitive to fluctuations in environmental temperature which can result in deviations from Tc. Furthermore, heart rate is highly reflective of the body's metabolic demands, which in turn alters thermoregulation and consequently Tc. In view of these, a non-invasive, accurate and practical method/device for monitoring of Tc continuously to prevent heat related injuries is highly desired.

### SUMMARY OF THE INVENTION

The above and other problems are solved and an advance in the art is made by provision of a system and method for continuous monitoring of core body temperature (Tc) of a user. Estimation of Tc is performed continuously with a Tc prediction model using the measured physiological data of the user where the effect of heart rate and external environmental temperature on the auditory canal temperature of the user are taken into account.

This invention has many benefits and advantages, such as non-invasive, accurate, portable, user friendly, reusable, less costly than invasive methods, and suitable for outdoor use. Particularly, this invention enhances the accuracy and reliability of Tc estimation as the effect of heart rate and external environmental temperature on the auditory canal temperature of the user are taken into account. This invention is safe and easy to use as the sensors are located near the opening of the auditory canal (i.e. away from eardrum) and external auricle. Hence, comparing to invasive methods, this invention minimizes user discomfort and mitigates the risk during the insertion of invasive probes. Furthermore, physiological data measurements can be wirelessly transmitted to the analysis unit (for Tc estimation) which can be a mobile phone that we carry with us every day. The operation of the system is simple, fast, easy to use by anyone and suitable for outdoor use due to its portability. By monitoring of Tc continuously, personnel can be withdrawn from operations before critical Tc is reached (about 40° C.) thereby enhancing safety. Furthermore, this invention is versatile as it can be embedded or integrated into an earphone with audio functionalities where continuous feedback via audio or video functionalities may be provided.

A system for continuous monitoring of core body temperature (Tc) of a user is provided. The system comprising: (1) a detection unit to be worn in the user's ear for measuring physiological data of the user by a plurality of sensors installed at the detection unit wherein the physiological data to be measured comprise first auditory canal temperature (T_{ac1}), second auditory canal temperature (T_{ac2}), external auricle temperature (Tₑₐ) and heart rate (HR) of the user; and (2) an analysis unit connected to the detection unit via a communication link for computing Tc of the user with a prediction model using the physiological data measured by the detection unit where the effect of heart rate and external environmental temperature on auditory canal temperature of the user are taken into account. An over-heating state is detected when the computed Tc of the user is above a threshold level. Preferably, the threshold level is 40° C.

The plurality of sensors comprising: a first temperature sensor for measuring the T_{ac1}, a second temperature sensor for measuring the T_{ac2}, a third temperature sensor for measuring the Tₑₐ, and an optical sensor for measuring the HR. Preferably, the first and second temperature sensors are thermocouple sensors. Preferably, the third temperature sensor is an infrared sensor. Preferably, the optical sensor is a photoplethysmogram sensor. The physiological data of the user are measured repeatedly according to a pre-defined time interval so that Tc of the user can be monitored continuously.

The detection unit comprising: an earbud to fit to the user's ear; a first extension member extends from the earbud for insertion into auditory canal of the user's ear wherein the first temperature sensor, the second temperature sensor and the optical sensor are installed at the first extension member for measuring the T_{ac1}, T_{ac2} and HR respectively; a second extension member extends from the earbud and in contact with the concha part of the user's ear wherein the third temperature sensor is installed at the second extension member for measuring the Tₑₐ; and a control module for receiving and sending the measured physiological data to the analysis unit, and alerting the user when the over-heating state is detected. The detection unit may further comprise an elastic member for sealing the auditory canal thereby minimising air exchange between the auditory canal and external environment.

The second extension member has an auricular hook structure to encircle around the back of the user's ear where the third temperature sensor is installed at a position in contact with the eminence of concha of the user's ear. Alternatively, the second extension member has an elongate structure extends to the cymba concha of the user's ear where the third temperature sensor is installed at a position in contact with the cymba concha.

The analysis unit comprising a data processing module for receiving the physiological data measured by the detection unit and computing Tc of the user with the prediction model using the physiological data where the effect of heart rate and external environmental temperature on auditory canal temperature of the user are taken into account. The analysis unit further comprising: a user interface for displaying the computed Tc and/or the measured physiological data of the user, and allowing the user to change Tc computation parameters; and a memory for storing the computed Tc and/or the measured physiological data of the user. The analysis unit can be in the form of a smart device installed with a software application to compute Tc of the user and display the computed Tc and/or the measured physiological data of the user.

The prediction model is a random forest prediction model which utilises a machine learning algorithm to compute Tc of the user with an acceptable mean bias of less than ±0.27° C. where the measured physiological data are used to derive a decision tree to predict Tc of the user.

Alternatively, the prediction model is a linear regression prediction model which uses a formula and the measured physiological data to compute Tc of the user where the formula is:

15.4299+3.6506T_{ac1}−3.1375T_{ac2}+0.0682Tₑₐ+0.0037HR

Alternatively, the prediction model is a polynomial regression prediction model of degree 2 which uses a formula and the measured physiological data to compute Tc of the user where the formula is:

−77.6520+82.9429T_{ac1}−75.4587T_{ac2}−2.4982Tₑₐ−0.0320HR−6.1514T_{ac1}²+8.4253(T_{ac1}×T_{ac2})+1.7738(T_{ac1}×Tₑₐ)+0.0332(T_{ac1}×HR)−2.4006T_{ac2}²−1.6639(T_{ac2}×Tₑₐ)−0.0357(T_{ac2}×HR)−0.0355Tₑₐ²+0.0040(Tₑₐ×HR)−0.0001HR².

A method for continuous monitoring of core body temperature (Tc) of a user is provided. The method comprising: measuring physiological data of the user by a plurality of sensors installed at a detection unit to be worn in the user's ear wherein the physiological data to be measured comprise first auditory canal temperature (T_{ac1}), second auditory canal temperature (T_{ac2}), external auricle temperature (Tₑₐ) and heart rate (HR) of the user; sending the measured physiological data to an analysis unit connected to the detection unit via a communication link; computing Tc of the user by the analysis unit with a prediction model using the physiological data measured by the detection unit where the effect of heart rate and external environmental temperature on auditory canal temperature of the user are taken into account; determining an over-heating state when the computed Tc of the user is above a threshold level; and generating a warning signal to alert the user when the over-heating state is determined. The method further comprising: displaying the computed Tc and/or the measured physiological data on the analysis unit; and storing the computed Tc and/or the measured physiological data in the analysis unit.

The step of measuring the physiological data of the user is repeated according to a pre-defined time interval so that Tc of the user can be monitored continuously.

The prediction model of the Tc computation step is a random forest prediction model which utilises a machine learning algorithm to compute Tc of the user with an acceptable mean bias of less than ±0.27° C. where the measured physiological data are used to derive a decision tree to predict Tc of the user.

### BRIEF DESCRIPTION OF THE DRAWINGS

The above and other features and advantages of this invention are described in the following detailed description of preferred embodiments with reference to the below figures:

FIG. 1 shows a system for monitoring Tc of a user continuously in accordance with an embodiment of this invention.

FIG. 2 shows a detection unit in accordance with a first embodiment of this invention.

FIG. 3 shows a detection unit in accordance with a second embodiment of this invention.

FIG. 4 show the front view (A) and back view (B) of the auricle of an ear.

FIG. 5 is a cross-sectional view of an ear showing the auditory canal.

FIG. 6 shows a flowchart of a method for monitoring Tc of a user continuously in accordance with an embodiment of this invention.

FIG. 7 are Bland-Altman plots comparing agreement between (A) Tₗᵢₙ and T_{gi}, (B) T_{poly} and T_{gi}, and (C) T_{rf} and T_{gi} during baseline with mean bias (solid line), ideal limits of agreement, LOAᵢ (±0.27° C.; dotted lines) and maximum limits of agreement, LOAₘₐₓ (±0.40° C.; dashed lines).

FIG. 8 are Bland-Altman plots comparing agreement between (A) Tₗᵢₙ and T_{gi}, (B) T_{poly} and T_{gi}, and (C) T_{rf} and T_{gi} during PAH with mean bias (solid line), LOAᵢ (±0.27° C.; dotted lines) and LOAₘₐₓ (±0.40° C.; dashed lines).

FIG. 9 are Bland-Altman plots comparing agreement between (A) Tₗᵢₙ and T_{gi}, (B) T_{poly} and T_{gi}, and (C) T_{rf} and T_{gi} during RUN with mean bias (solid line), LOA (±0.27° C.; dotted lines) and LOAₘₐₓ (±0.40° C.; dashed lines).

FIG. 10 are Bland-Altman plots comparing agreement between (A) Tₗᵢₙ and T_{gi}, (B) T_{poly} and T_{gi}, and (C) T_{rf} and T_{gi} during WALK with mean bias (solid line), LOAᵢ (±0.27° C.; dotted lines) and LOAₘₐₓ (±0.40° C.; dashed lines).

FIG. 11 are Bland-Altman plots comparing agreement between (A) Tₗᵢₙ and T_{gi}, (B) T_{poly} and T_{gi}, and (C) T_{rf} and T_{gi} during recovery with mean bias (solid line), LOAᵢ (±0.27° C.; dotted lines) and LOAₘₐₓ (±0.40° C.; dashed lines).

### DETAILED DESCRIPTION OF THE INVENTION

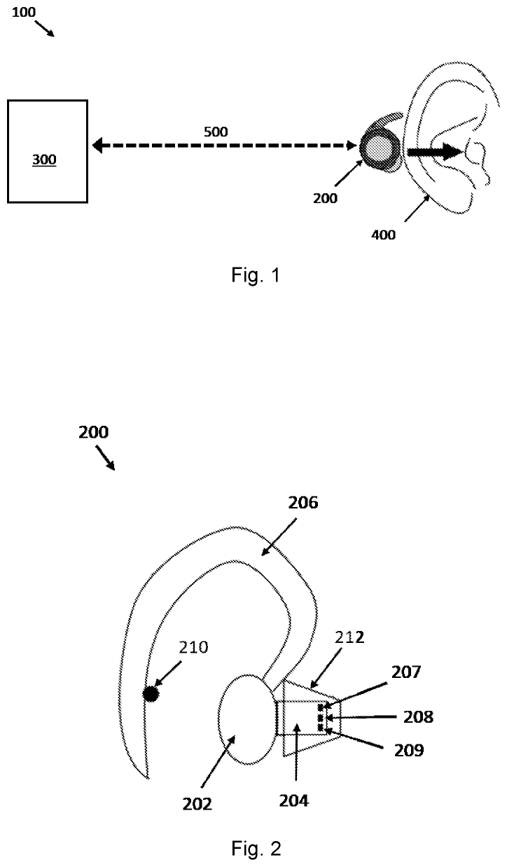

FIG. 1 illustrates system 100 for continuous monitoring of core body temperature (Tc) of a user in a non-invasive manner so that an over-heating state of the user can be detected in which the computed Tc is above a threshold level, such as 40° C. The threshold level is changeable based on an individual requirement. System 100 comprises detection unit 200 and analysis unit 300 connected to each other through communication link 500, which can be a wireless communication (e.g. Bluetooth) or a wired communication. Detection unit 200 is an ear-based device to be worn in the user's ear 400 for measuring physiological data of the user. Detection unit 200 can be worn like an earphone for a long period of time without feeling discomfort due to its small size and lightweight. On the other hand, analysis unit 300 can be in a form of smart device (e.g. mobile phone) installed with software application to compute Tc of the user efficiently and rapidly, and provide a user-friendly interface to display the computed Tc and/or measured physiological data of the user.

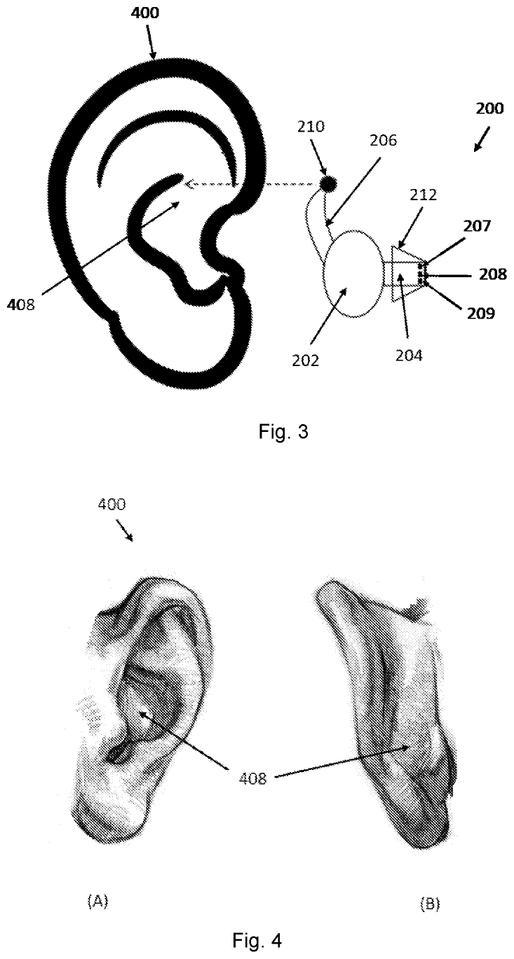

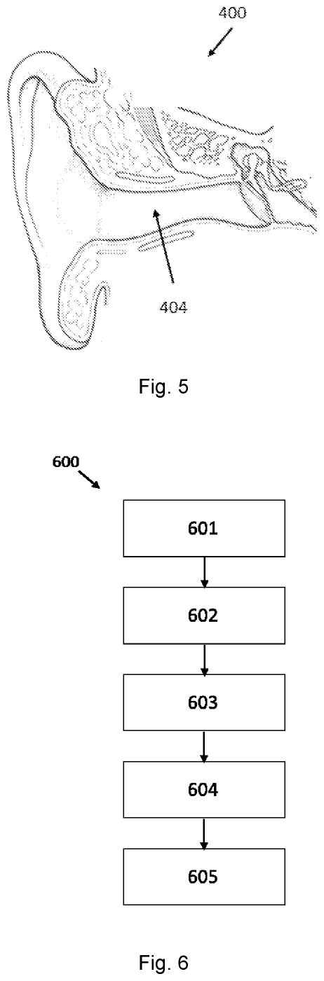

FIGS. 2 and 3 showing two different designs of detection unit 200. FIG. 4 shows the front view and back view of human ear 400. FIG. 5 is a cross-sectional view of human ear 400 showing auditory canal 404. Detection unit 200 comprises earbud 202 to fit to the user's ear, first and second extension members 204, 206 that extend from earbud 202, and a control module (not shown). A plurality of sensors 207, 208, 209 and 210 are installed at detection unit 200 for measuring physiological data of the user, which include first auditory canal temperature (T_{ac1}) measured by first temperature sensor 207, second auditory canal temperature (T_{ac2}) measured by second temperature sensor 208, external auricle temperature (Tₑₐ) measured by third temperature sensor 210, and heart rate (HR) of the user measured by optical sensor 209. It is possible that more sensors may be used to obtain more physiological variables depending on the algorithm/formula used for Tc estimation.

Detection unit 200 may further comprise elastic member 212 for sealing auditory canal 404 so that air exchange between auditory canal 404 and external environment can be minimised. Elastic member 212 is made of a skin-friendly material, such as silicone, rubber or other suitable materials, so that detection unit 200 can be worn comfortably for long period. Elastic member 212 is also replaceable with a suitable size that is best fit for the user, such as different sizes for adults and children. As detection unit 200 is reusable by the same or different user, it should be made by a material that can withstand a sterilising process as cleaning is required after use. Detection unit 200 may also be integrated into an earphone with audio functionality.

First extension member 204 is a short elongate structure (e.g. 8 mm long) extends from earbud 202 for insertion into auditory canal 404 of the user. First temperature sensor 207, second temperature sensor 208 and optical sensor 209 are installed at first extension member 204 at appropriate locations for measuring T_{ac2}, T_{ac2} and HR of the user respectively in auditory canal 404. For example, sensors 207, 208 and 209 may be installed around the end part of first extension member 204 as shown in FIGS. 2 and 3.

Second extension member 206 extends from earbud 202 and in contact with concha part 408 of the user's ear 400. FIG. 2 shows a first design of second extension member 206 which has an auricular hook structure to encircle around the back of the user's ear 400. Third temperature sensor 210 is installed at the auricular hook structure and in contact with the back of concha part 408, i.e. the eminence of concha (see FIG. 4(B)) for measuring external temperature of the user's ear 400 (i.e. external auricle temperature Tₑₐ). FIG. 3 shows a second design of second extension member 206 which has a shorter elongate structure than the auricular hook structure where third temperature sensor 210 is installed around the end part and in contact with the front of concha region 408, i.e. cymba concha (see FIG. 4(A)) for measuring external temperature of the user's ear 400 (i.e. external auricle temperature Tₑₐ).

Each of temperature sensors 207, 208, 210 can be a thermocouple sensor or an infrared sensor. Optical sensor 209 can be a photoplethysmogram sensor. The physiological data of T_{ac1}, T_{ac2}, Tₑₐ and HR obtained by detection unit 200 will be sent to analysis unit 300 for Tc computation. T_{ac1}, T_{ac2}, Tₑₐ and HR are measured repeatedly according to a pre-defined time interval (e.g. every 1 minute) so that Tc of the user can be monitored continuously. The time interval is changeable based on individual requirement and/or external environment conditions.

The accuracy of Tc estimation increases significantly when the user's ear is properly sealed and insulated, or when the ear is maintained in a tight and controlled thermal condition. However, sealing or insulation of the user's ear completely is neither a desirable nor feasible option for most heat-exposed occupations as this may result in the accumulation of heat during physical activity and thus affect accuracy of the method and may also make users feel uncomfortable. Thus, instead of sealing the ear completely, this invention seeks to enhance Tc accuracy by accounting for the changes in ambient temperature and heart rate of the user during the estimation of Tc. In this context, T_{ac1}, T_{ac2}, Tₑₐ and HR are measured concurrently and used for computation of Tc with greater accuracy. Therefore, Tc of the user can be accurately monitored regardless of the environment and activity of the user.

Earbud 202 is a small housing configured to be securely fitted to the opening of auditory canal 404 of the user's ear 400. Preferably, the control module of detection unit 200 is disposed within earbud 202.

The control module receives the measured physiological data T_{ac2}, T_{ac2}, Tₑₐ and HR of the user and send them to analysis unit 300 through communication link 500. The person who carrying analysis unit 300 can communicate or alert the user when an over-heating state is detected by analysis unit 300. Alternatively, the control module of detection unit 200 may also alert the user via an audio function when an over-heating state is detected by analysis unit 300, or a fault in the communication between detection unit 200 and analysis unit 300 is detected. It is also possible that detection unit 200 has an alarm to alert the user or people around the user with a speaker or a light-emitting diode (LED) when an over-heating state is detected by analysis unit 300.

Analysis unit 300 comprising a data processing module, a user-friendly interface, and a memory. The data processing module receives the measured physiological data T_{ac1}, T_{ac2}, Tₑₐ and HR from detection unit 200 and computes Tc of the user with a prediction model using the measured physiological data where the effect of heart rate and external environmental temperature on the auditory canal temperature of the user are taken into account. Preferably, the prediction model is a random forest prediction model which utilises a machine learning algorithm to compute Tc of the user with an acceptable low mean bias of less than ±0.27° C. where the measured physiological data are used to derive a decision tree to predict the Tc. The data processing module will generate and transmit a warning signal to detection unit 200 to alert the user when an over-heating state of the user is detected. The user interface can display the computed Tc and/or measured physiological data of the user (and any other information), and allow the user to change the Tc computation parameters. The memory is used for storing the computed Tc and/or measured physiological data of the user.

FIG. 6 illustrates a flowchart of a method for continuous monitoring of Tc of a user using system 100 as described above. Method 600 comprising the following steps. In step 601, physiological data T_{ac1}, T_{ac2}, Tₑₐ and HR of the user are measured by a plurality of sensors 207, 208, 209, 210 installed at detection unit 200 to be worn in the user's ear. In step 602, the measured physiological data T_{ac1}, T_{ac2}, Tₑₐ and HR are sent to analysis unit 300 which in communication with detection unit 200 through communication link 500. In step 603, Tc of the user is computed by analysis unit 300 with a Tc prediction model using the measured physiological data where the effect of heart rate and external environmental temperature on the auditory canal temperature of the user are taken into account. In step 604, an over-heating state of the user is determined when the computed Tc is above a threshold level (such as 40° C.). In step 605, a warning signal is generated to alert the user when an over-heating state is determined. The method may further comprising the steps of: displaying the computed Tc and/or the measured physiological data on the analysis unit; and storing the computed Tc and/or the measured physiological data in the analysis unit. The above steps are repeated continuously according to a pre-defined time interval (e.g. every 1 minute) so that Tc of the user can be monitored continuously. The Tc prediction model of the method can be a random forest prediction model, a linear regression prediction model, or a polynomial regression prediction model of degree 2, which will be described below. The random forest prediction model is the preferred model as it has an acceptable mean bias of less than ±0.27° C. and a relatively small mean absolute error.

The measured physiological data T_{ac1}, T_{ac2}, Tₑₐ and HR of the user were utilised to develop three potential Tc prediction models: (1) random forest prediction model (T_{rf} model), (2) linear regression prediction model (T_{rf} model), and (3) polynomial regression prediction model of degree 2 (T_{poly} model). To refine the invention, the three developed prediction models were validated against gastrointestinal temperature (TO derived from a telemetric pill (corresponding to Tc of the user). In doing so, the most accurate and reliable Tc prediction model across varying modes of heating can be identified. Twenty healthy aerobically fit males (age=25±3 years, body mass index (BMI)=21.7±1.8, body fat=12±3%, maximal aerobic capacity (VO₂ₘₐₓ)=64±7 ml/kg/min) participated in this study. Participants performed a VO₂max test followed by three experimental trials: a passive heating trial (PAH), a running trial (RUN), and a brisk walking trial (WALK). Among the three evaluated prediction models, T_{rf} model is the most ideal prediction model across all measurement phases.

Maximal Aerobic Capacity (VO₂ₘₐₓ) Test:

An incremental exercise protocol was used to measure each participant's VO₂ₘₐₓ. In the first phase, participants performed a treadmill run at four different speeds, with an initial speed that was 1 km/h slower than their expected 10 km race pace. Treadmill speed was increased by 1 km/h every 3 min, for a total duration of 12 min. Following a 5 min rest, participants proceeded to the second phase which consisted of a treadmill run at a fixed speed of moderate intensity, with an initial elevation of 1%. Treadmill elevation was increased by 1% every min until volitional exhaustion was reached. VO₂ₘₐₓ was established as the mean VO₂ during the final minute prior to termination of the test.

Experimental Trials:

All participants followed a similar diet and repeat any physical activity performed 24-hour prior to each experimental trial. Urine SG was measured to ensure that participants adequately hydrated prior to commencement of each session (urine SG<1.025). Participants' T_{gi} and HR were monitored using an ingestible telemetric sensor and chest-based monitor respectively. The temperature sensor was either ingested 8-10 hours before each session or rectally inserted upon arrival at the trial site. T_{ac1}, T_{ac2}, Tₑₐ and HR were continuously recorded by an ear-based detection unit. Participants were provided with 2 g/kg body mass of water maintained at 26° C., every 15 min. A metabolic cart was used to measure VO₂ at specific time points during RUN and WALK.

Passive Heating Trial (PAH):

Participants donned running shorts and completed a 10 min seated baseline in an air-conditioned laboratory environment (Dry Bulb Temperature: T_{db}=21.6±0.5° C., Relative Humidity: RH=68±3%, Wet Bulb Globe Temperature: WBGT=19.2±0.5° C.). Following which, participants immersed themselves up to chest level in an inflatable tub containing water that was maintained at 42.0±0.3° C. by an external heating unit. Light facial fanning was applied to minimise participant discomfort. Participants were passively heated until either T_{gi} of 39.5° C. or total duration of 60 min was reached. Subsequently, participants underwent a seated recovery until T_{gi} returned below 38.0° C. As a safety precaution, facial fanning was also employed during recovery.

Running Trial (RUN) and Brisk Walking Trial (WALK):

Participants donned running attire with sports shoes and completed a 10 min seated baseline in a controlled environmental chamber (T_{db}=30.0±0.2° C., RH=71±2%, WBGT=27.1±0.3° C.). During RUN, participants ran on a motorised treadmill at a speed that corresponded to 70±3% of their VO₂ₘₐₓ. During WALK, participants performed a treadmill walk at 6 km/h with an elevation of 7%. In both trials, exercise was terminated when T_{gi} reached 39.5° C. Participants that did not achieve the target T_{gi} within a 60 min duration underwent an extended exercise phase. This consisted of a treadmill walk at a speed of 6 km/h with an elevation of 1%, for a maximum duration of 30 min. Subsequently, participants underwent a seated recovery until T_{gi} returned below 38.0° C.

Model Development:

Physiological data were collected from two thermocouple sensors (for T_{ac1} and T_{ac2}), one infrared sensor (for Tₑₐ) and one photoplethysmogram sensor (for HR) over the course of the baseline phase (10 min), exercise/heating phase and recovery phase (until participant's T_{gi} returned below 38.0° C.). Measurements for T_{ac1}, T_{ac2}, Tₑₐ and HR were logged in one second intervals while measurements for T_{gi} were logged every 15 seconds.

The Tₗᵢₙ model was generated to predict T_{gi} based on inputs from T_{ac1}, T_{ac2}, Tₑₐ and HR as follows (presented to the nearest four decimal place):

15.4299+3.6506T_{ac1}−3.1375T_{ac2}+0.0682Tₑₐ+0.0037HR

The T_{poly} model was generated to predict T_{gi} based on inputs from T_{ac1}, T_{ac2}, Tₑₐ and HR as follows (presented to the nearest four decimal place):

−77.6520+82.9429T_{ac1}−75.4587T_{ac2}−2.4982Tₑₐ−0.0320HR−6.1514T_{ac1}²+8.4253(T_{ac1}×T_{ac2})+1.7738(T_{ac1}×Tₑₐ)+0.0332(T_{ac1}×HR)−2.4006T_{ac2}²−1.6639(T_{ac2}×Tₑₐ)−0.0357(T_{ac2}×HR)−0.0355Tₑₐ²+0.0040(Tₑₐ×HR)−0.0001HR²

For example, when T_{ac1}=37.0° C., T_{ac}2=36.9° C., Tₑₐ=36.5° C. and HR=70 bpm, the predicted T_{gi} by Tₗᵢₙ and T_{poly} models are as follows:

Tₗᵢₙ model=15.4299+(3.6506×37.0)−(3.1375×36.9)+(0.0682×36.5)+(0.0037×70)=37.48° C. a)

T_{poly} model=−77.6520+(82.9429×37.0)−(75.4587×36.9)−(2.4982×36.5)−(0.0320×70)−(6.1514×(37.0)²)+(8.4253×(37.0×36.9))+(1.7738×(37.0×36.5))+(0.0332×(37.0×70))−(2.4006×(36.9)²)−(1.6639×(36.9×36.5))−(0.0357×(36.9×70))−(0.0355×(36.5)²)+(0.0040×(36.5×70))−(0.0001×(70)²)=37.08° C. b)

As for the T_{rf} model, a randomly selected subset of T_{ac1}, T_{ac2}, Tₑₐ, HR, and their derivatives were used by machine learning to derive a decision tree which produced a value with low mean squared error in relation to the corresponding T_{gi}. This process was repeated with different sets of subsets, and the final value was derived from the mean of the predicted values. As the T_{rf} model has a low overall biasness, it is highly stable when new data is introduced and is robust with both categorical and numerical data. The one-hot encoding technique was employed to convert categorical variables, such as participants, mode of training, and phase of exercise into columns of numerical binary data. Therefore, if a data point is at baseline, it will have the value ‘1’ in the baseline column and ‘0’ in the other columns. This step is done in Python using the function get_dummies.

Furthermore, feature scaling was used to scale all numerical values in the dataset to ensure that all features were evaluated with equal importance, regardless of their absolute numerical value. To do so, Sci-kit-Learn's StandardScaler class was employed. The RandomForestRegressor class of the sklearn.ensemble library was used to solve regression problems in the T_{rf} model. Among the parameters one can employ to configure a T_{rf} model, the most crucial parameter is the n_estimators parameter. This value defines the number of trees in the T_{rf} model. In the developed algorithm, n_estimators=100 was chosen to achieve a balance of accuracy and computational resources.

A total of 16 participants were utilized to train each prediction model, which were then optimized by a rolling average filter. This filtered prediction was compared against data from the remaining four participants to evaluate model validity. Furthermore, to assess the reliability of the T_{rf} model, a five-fold average analysis was performed wherein each data fold (Fold-1 to Fold-5) consisted of a different combination of 16 participants for model training and four participants for model validity testing respectively.

Statistical Analysis:

Normality of data was assessed using a Shapiro-Wilk test. Two-tailed paired t-test was performed to assess for differences between trials. Pearson's correlation coefficient (r) was used to evaluate the degree of correlation between T_{gi} and each of the three prediction models. The degree of correlation was determined as follows: very strong (r>0.90), strong (r=0.70 to <0.90), moderate (r=0.50 to <0.70), low (r=0.30 to <0.50) and negligible (r<0.30). Bland-Altman plots were used to assess for the agreement between the T_{gi} data derived from the telemetric pill and the outputs from the three prediction models. Furthermore, the corresponding values for mean bias, 95% confidence intervals (CI), mean absolute error (MAE) and mean absolute percentage error (MAPE) were calculated for each prediction model. All data were presented in mean±SD and a 0.05 level of significance was used for all statistical analyses. Statistical significance was represented as follows: *: p<0.05, **: p<0.01, ***: p<0.001. The following criterion were used to determine the validity of the prediction models to predict T_{gi}: (a) mean bias <±0.27° C., and (b) 95% CI within ±0.40° C.

Results:

The validity measures (mean bias, 95% CI, MAE and MAPE) and correlation for each prediction model (Tₗᵢₙ, T_{poly} and T_{rf}) are depicted in Table 1 below. The three prediction models were evaluated against T_{gi} measured using gold standard temperature capsule in five separate phases as follows: a) baseline rest, b) passive heating, c) exercise run, d) exercise walk, and e) seated recovery. Mean bias was within the validity criterion of <±0.27° C. during all measurement phases in T_{rf} model (−0.20 to 0.13° C.) but not in Tₗᵢₙ model (−0.63 to 0.68° C.) and T_{poly} model (−0.37 to 0.64° C.). The 95% CI in the T_{rf} model was also within the validity criterion of ±0.4° C. during baseline (−0.35 to 0.26° C.) but not in other measurement phases. The 95% CI for Tₗᵢₙ and T_{poly} models exceeded the validity criterion during all measurement phases. Both MAE and MAPE appeared to be smaller in the T_{rf} model as compared to Tₗᵢₙ and T_{poly} models. During baseline, Tₗᵢₙ model (r=0.677, p<0.01) and T_{poly} model (r=0.591, p<0.01) were observed to be moderately correlated with T_{gi} while the correlation between T_{rf} model and T_{gi} was negligible (r=0.225, p<0.01). During exercise and heating, T_{rf} model demonstrated a very strong correlation with T_{gi} (r=0.902 to 0.933, p<0.01) while Tₗᵢₙ model (r=0.708 to 0.955, p<0.01) and T_{poly} model (r=0.865 to 0.957, p<0.01) exhibited a strong to very strong correlation with T_{gi}. All prediction models were observed to be strongly correlated with T_{gi} during recovery (Tₗᵢₙ: r=0.708, p<0.01, T_{poly}: r=0.742, p<0.01, Tₗᵢₙ: r=0.819, p<0.01).

TABLE 1
A summary of validity measures and correlation to
compare Tlin, Tpoly and Trf prediction models.

| | | Tₗᵢₙ | T_{poly} | T_{rf} |
|---|---|---|---|---|
| a) Baseline: | Mean bias (° C.) | 0.36 ± 0.11 | 0.23 ± 0.20^{‡} | −0.05 ± 0.16^{‡} |
| | 95% CI (° C.) | 0.14 to 0.59 | −0.16 to 0.61 | −0.35 to 0.26^{‡} |
| | MAE (° C.) | 0.36 ± 0.11 | 0.26 ± 0.15 | 0.14 ± 0.09 |
| | MAPE (%) | 0.98 ± 0.31 | 0.71 ± 0.40 | 0.37 ± 0.24 |
| | r | 0.677** | 0.591** | 0.225** |
| b) Heating | Mean bias (° C.) | 0.68 ± 0.40 | 0.64 ± 0.38 | −0.20 ± 0.38^{‡} |
| (PAH): | 95% CI (° C.) | −0.11 to 1.47 | −0.10 to 1.38 | −0.94 to 0.54 |
| | MAE (° C.) | 0.70 ± 0.37 | 0.65 ± 0.34 | 0.34 ± 0.27 |
| | MAPE (%) | 1.85 ± 1.00 | 1.73 ± 0.92 | 0.88 ± 0.68 |
| | r | 0.874** | 0.901** | 0.909** |
| c) Exercise | Mean bias (° C.) | −0.01 ± 0.39^{‡} | −0.08 ± 0.30^{‡} | −0.15 ± 0.27^{‡} |
| (RUN): | 95% CI (° C.) | −0.77 to 0.75 | −0.66 to 0.51 | −0.69 to 0.39 |
| | MAE (° C.) | 0.33 ± 0.20 | 0.25 ± 0.17 | 0.25 ± 0.20 |
| | MAPE (%) | 0.86 ± 0.54 | 0.66 ± 0.46 | 0.64 ± 0.50 |
| | r | 0.955** | 0.957** | 0.933** |
| d) Exercise | Mean bias (° C.) | 0.15 ± 0.38^{‡} | 0.11 ± 0.28^{‡} | 0.13 ± 0.23^{‡} |
| (WALK): | 95% CI (° C.) | −0.58 to 0.89 | −0.43 to 0.66 | −0.31 to 0.58 |
| | MAE (° C.) | 0.35 ± 0.21 | 0.25 ± 0.17 | 0.22 ± 0.14 |
| | MAPE (%) | 0.92 ± 0.55 | 0.65 ± 0.47 | 0.58 ± 0.38 |
| | r | 0.708** | 0.865** | 0.902** |
| e) Recovery: | Mean bias (° C.) | −0.63 ± 0.45 | −0.37 ± 0.43 | −0.06 ± 0.36^{‡} |
| | 95% CI (° C.) | −1.50 to 0.25 | −1.22 to 0.48 | −0.77 to 0.65 |
| | MAE (° C.) | 0.65 ± 0.42 | 0.44 ± 0.36 | 0.27 ± 0.25 |
| | MAPE (%) | 1.65 ± 1.06 | 1.14 ± 0.92 | 0.70 ± 0.63 |
| | r | 0.708** | 0.742** | 0.819** |

^{‡}indicates within validity criterion: a) mean bias < ±0.27° C. or 95% CI within ±0.40° C.

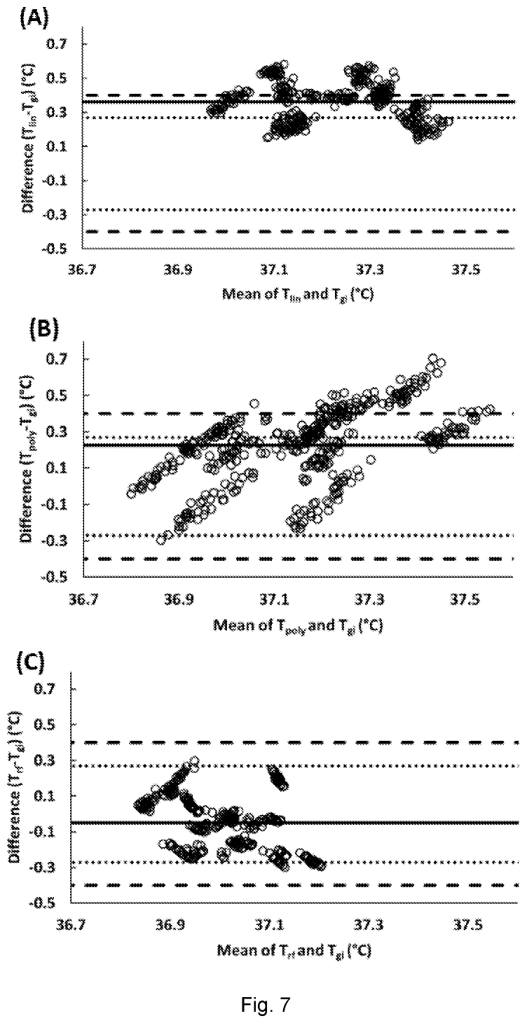

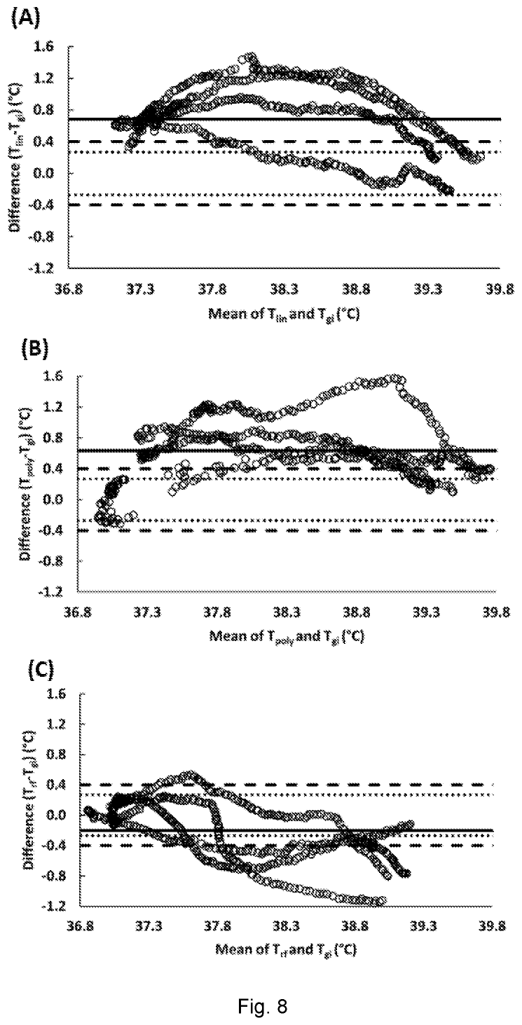

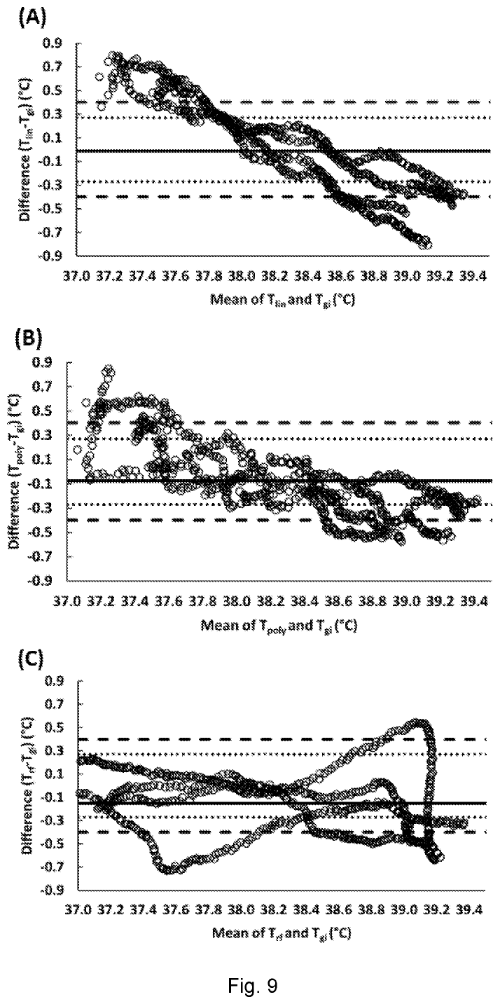

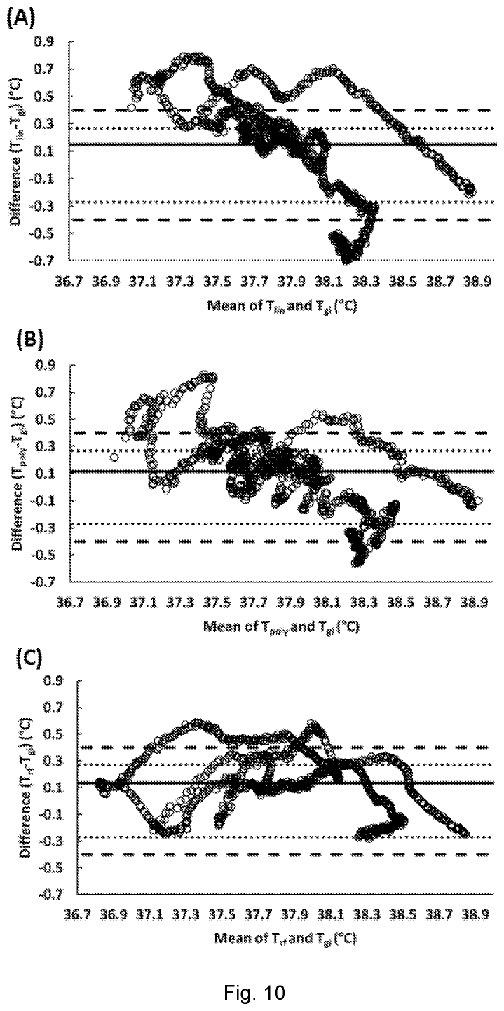

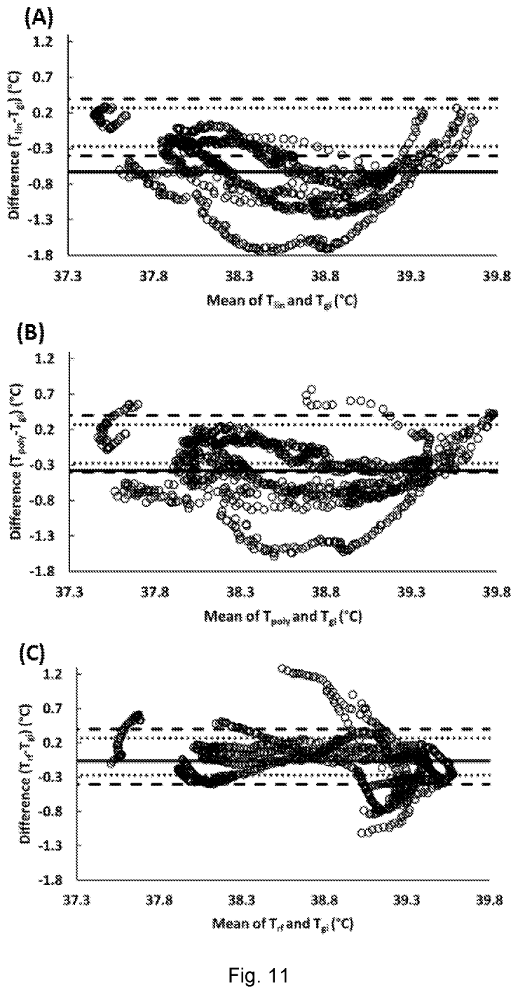

During baseline, 429 paired data points were assessed for T_{rf} model with all data points observed to be within LOAₘₐₓ (FIG. 7 (C)). In turn, 440 paired data points were assessed for Tₗᵢₙ and T_{poly} models with 62% and 80% of data points found to be within LOAₘₐₓ (FIGS. 7 (A) and (B)) respectively. During PAH, 537 paired data points were assessed for Tₗᵢₙ, T_{poly} and T_{rf} models with 25%, 23% and 66% of data points found to be within LOAₘₐₓ (FIGS. 8 (A), (B) and (C)) respectively. During RUN, 720 paired data points were assessed for Tₗᵢₙ, T_{poly} and T_{rf} models with 65%, 77% and 73% of data points found to be within LOAₘₐₓ (FIGS. 9 (A), (B) and (C)) respectively. During WALK, 887 paired data points were assessed for Tₗᵢₙ, T_{poly} and T_{rf} models with 63%, 82% and 85% of data points found to be within LOAₘₐₓ (FIGS. 10 (A), (B) and (C)) respectively. During recovery, 1004 paired data points were assessed for Tₗᵢₙ, T_{poly} and T_{rf} models with 33%, 54% and 79% of data points found to be within LOAₘₐₓ (FIGS. 11 (A), (B) and (C)) respectively.

A five-fold average for the T_{rf} model was assessed for validity measures (mean bias, 95% CI, MAE and MAPE) and correlation in each separate phase of the trial (baseline, PAH, RUN, WALK and recovery), as shown in Table 2 below. Overall, 18897 paired data points were assessed in the five-fold average for the T_{rf} model. Mean bias was within the validity criterion (<±0.27° C.) across all phases of the trial (−0.26 to 0.01° C.). Further, 95% CI was close to the validity criterion during baseline (−0.39 to 0.41° C.) but exceeded the range of acceptability in the remaining trial phases (95% CI>±0.40° C.). The MAE appeared to be small during baseline (0.17±0.12° C.) and WALK (0.28±0.25° C.). Finally, the five-fold average of T_{rf} model demonstrated a strong correlation with T_{gi} in all trial phases (r=0.780 to 0.855, p<0.01) except during baseline (r=0.332, p<0.01).

TABLE 2
Five-fold average analysis of validity measures
to assess reliability of the T_{rf} model.

| | | Five-fold average T_{rf} |
|---|---|---|
| a) Baseline: | Mean bias (° C.) | −0.01 ± 0.20^{‡} |
| | 95% CI (° C.) | −0.39 to 0.41 |
| | MAE (° C.) | 0.17 ± 0.12 |
| | MAPE (%) | 0.45 ± 0.32 |
| | r | 0.332** |
| b) Heating | Mean bias (° C.) | −0.25 ± 0.49^{‡} |
| (PAH): | 95% CI (° C.) | −1.20 to 0.70 |
| | MAE (° C.) | 0.40 ± 0.37 |
| | MAPE (%) | 1.05 ± 0.95 |
| | r | 0.808** |
| c) Exercise | Mean bias (° C.) | −0.26 ± 0.44^{‡} |
| (RUN): | 95% CI (° C.) | −1.12 to 0.61 |
| | MAE (° C.) | 0.38 ± 0.34 |
| | MAPE (%) | 1.00 ± 0.86 |
| | r | 0.824** |
| d) Exercise | Mean bias (° C.) | −0.15 ± 0.34^{‡} |
| (WALK): | 95% CI (° C.) | −0.82 to 0.52 |
| | MAE (° C.) | 0.28 ± 0.25 |
| | MAPE (%) | 0.74 ± 0.64 |
| | r | 0.780** |
| e) Recovery: | Mean bias (° C.) | 0.01 ± 0.45^{‡} |
| | 95% CI (° C.) | −0.87 to 0.88 |
| | MAE (° C.) | 0.34 ± 0.29 |
| | MAPE (%) | 0.89 ± 0.74 |
| | r | 0.855** |

^{‡}indicates within validity criterion: a) mean bias < ±0.27° C. or 95% CI within ±0.40° C.

### CONCLUSIONS

All three Tₗᵢₙ, T_{poly} and T_{rf} models are largely able to predict T_{gi} during the exercise phases of the RUN and WALK trials. This was corroborated by acceptable mean biases of <±0.27° C. (Table 1). However, the results for Tₗᵢₙ and T_{poly} models during PAH and recovery appear to be poorer than T_{rf} model. It is known that auditory canal temperature (T_{ac}) is highly affected by environmental conditions. Furthermore, T_{ac} responds more quickly to Tc changes as compared to gastrointestinal temperature and/or rectal temperature. As such, the combinatorial effect of radiative heat from the water surface (environmental conditions) and a faster T_{ac} response to increasing Tc may have contributed to overestimation of Tgi during PAH and underestimation of Tgi during recovery by the Tₗᵢₙ and T_{poly} models.

The T_{rf} model is the most ideal model for prediction of T_{gi} across all measurement phases. Apart from achieving an acceptable mean bias of less than ±0.27° C. across all phases of the trial (−0.20 to 0.13° C.), T_{rf} model also has a small MAE in all measurement phases (0.14 to 0.25° C.) except during PAH (0.34±0.27° C.; Table 1). This indicates that mean positive and negative deviations from T_{gi} are relatively small when utilizing the T_{rf} model. Furthermore, T_{rf} model has a smaller MAPE and narrower 95% CI as compared to Tₗᵢₙ and T_{poly} models across all trial phases (Table 1). In turn, the percentage of paired data points found to be within the set LOAₘₐₓ (±0.40° C.) were found to be greater in T_{rf} model (FIGS. 7 to 11). Taken together, T_{rf} model is better and able to correct for changes in environmental conditions and differences in thermal inertia between measurement sites. As such, the T_{rf} model is able to predict T_{gi} more accurately than the Tₗᵢₙ and T_{poly} models in all trials and/or measurement phases.

To assess the reliability of the T_{rf} model, a five-fold average analysis was performed. Overall, the five-fold average of the T_{rf} model demonstrated acceptable mean biases across all trial phases (−0.26 to 0.01° C., Table 2). This appears to be in line with the initial single-fold analysis of T_{rf} (mean bias <±0.27° C., Table 1). As such, the reliability of T_{rf} model can be observed from its consistent performance across the five folds of analysis. During baseline, 95% CI was observed to be close to the validity criterion (−0.39 to 0.41° C., Table 2) thus indicating that the T_{rf} model is largely able to estimate T_{gi} during rest. As this invention and algorithm are designed with the intention of monitoring occupational heat strain, it is therefore worth noting that a relatively small mean bias error was observed during WALK (−0.15±0.34° C., Table 2). This suggests that the T_{rf} model displays a promising accuracy for monitoring of thermal strain during low to moderate intensity exertion, which is common in occupational settings. Taken together, the T_{rf} algorithm of this invention has a promising accuracy with mean bias values within the acceptable standards of ±0.27° C. for thermal heat strain monitoring.

1. A system for continuous monitoring of core body temperature (Tc) of a user, the system comprising:
a detection unit to be worn in the user's ear for measuring physiological data of the user by a plurality of sensors installed at the detection unit wherein the physiological data to be measured comprise first auditory canal temperature (T_{ac1}), second auditory canal temperature (T_{ac2}), external auricle temperature (Tₑₐ) and heart rate (HR) of the user; and
an analysis unit connected to the detection unit via a communication link for computing Tc of the user with a prediction model using the physiological data measured by the detection unit where the effect of heart rate and external environmental temperature on auditory canal temperature of the user are taken into account;
wherein an over-heating state is detected when the computed Tc of the user is above a threshold level.

2. The system of claim 1, wherein the plurality of sensors comprising:
a first temperature sensor for measuring the T_{ac1};
a second temperature sensor for measuring the T_{ac2};
a third temperature sensor for measuring the Tₑₐ; and
an optical sensor for measuring the HR.

3. The system of claim 2, wherein the detection unit comprising:
an earbud to fit to the user's ear;
a first extension member extends from the earbud for insertion into auditory canal of the user's ear wherein the first temperature sensor, the second temperature sensor and the optical sensor are installed at the first extension member for measuring the T_{ac1}, T_{ac2} and HR respectively;
a second extension member extends from the earbud and in contact with the concha part of the user's ear wherein the third temperature sensor is installed at the second extension member for measuring the Tₑₐ; and
a control module for receiving and sending the measured physiological data to the analysis unit, and alerting the user when the over-heating state is detected.

4. The system of claim 3, wherein the second extension member has an auricular hook structure to encircle around the back of the user's ear where the third temperature sensor is installed at a position in contact with the eminence of concha of the user's ear.

5. The system of claim 3, wherein the second extension member has an elongate structure extends to the cymba concha of the user's ear where the third temperature sensor is installed at a position in contact with the cymba concha.

6. The system of claim 3, wherein the detection unit further comprising:
an elastic member for sealing the auditory canal thereby minimising air exchange between the auditory canal and external environment.

7. The system of claim 1, wherein the analysis unit comprising:
a data processing module for receiving the physiological data measured by the detection unit and computing Tc of the user with the prediction model using the physiological data where the effect of heart rate and external environmental temperature on auditory canal temperature of the user are taken into account.

8. The system of claim 1, wherein the analysis unit further comprising:
a user interface for displaying the computed Tc and/or the measured physiological data of the user, and allowing the user to change Tc computation parameters; and
a memory for storing the computed T_{c} and/or the measured physiological data of the user.

9. The system of claim 1, wherein the prediction model is a random forest prediction model which utilises a machine learning algorithm to compute Tc of the user with an acceptable mean bias of less than ±0.27° C. where the measured physiological data are used to derive a decision tree to predict Tc of the user.

10. The system of claim 8, wherein the prediction model is a linear regression prediction model which uses a formula and the measured physiological data to compute Tc of the user, the formula is:
15.4299+3.6506T_{ac1}−3.1375T_{ac2}+0.0682Tₑₐ+0.0037HR.

11. The system of claim 8, wherein the prediction model is a polynomial regression prediction model of degree 2 which uses a formula and the measured physiological data to compute Tc of the user, the formula is:
−77.6520+82.9429T_{ac1}−75.4587T_{ac2}−2.4982Tₑₐ−0.0320HR−6.1514T_{ac1}²+8.4253(T_{ac1}×T_{ac2})+1.7738(T_{ac1}×Tₑₐ)+0.0332(T_{ac1}×HR)−2.4006T_{ac2}²−1.6639(T_{ac2}×Tₑₐ)−0.0357(T_{ac2}×HR)−0.0355Tₑₐ²+0.0040(Tₑₐ×HR)−0.0001HR².

12. The system of claim 1, wherein the analysis unit can be in the form of a smart device installed with a software application to compute Tc of the user and display the computed Tc and/or the measured physiological data of the user.

13. The system of claim 1, wherein the physiological data of the user are measured repeatedly according to a pre-defined time interval so that Tc of the user can be monitored continuously.

14. The system of claim 1, wherein the threshold level is 40° C.

15. The system of claim 2, wherein the first and second temperature sensors are thermocouple sensors.

16. The system of claim 2, wherein the third temperature sensor is an infrared sensor.

17. A method for continuous monitoring of core body temperature (Tc) of a user, the method comprising:
measuring physiological data of the user by a plurality of sensors installed at a detection unit to be worn in the user's ear wherein the physiological data to be measured comprise first auditory canal temperature (T_{ac1}), second auditory canal temperature (T_{ac2}), external auricle temperature (Tₑₐ) and heart rate (HR) of the user;
sending the measured physiological data to an analysis unit connected to the detection unit via a communication link;
computing Tc of the user by the analysis unit with a prediction model using the physiological data measured by the detection unit where the effect of heart rate and external environmental temperature on auditory canal temperature of the user are taken into account;
determining an over-heating state when the computed Tc of the user is above a threshold level; and
generating a warning signal to alert the user when the over-heating state is determined.

18. The method of claim 17, further comprising:
displaying the computed Tc and/or the measured physiological data on the analysis unit; and
storing the computed Tc and/or the measured physiological data in the analysis unit.

19. The method of claim 17, where the prediction model is a random forest prediction model which utilises a machine learning algorithm to compute Tc of the user with an acceptable mean bias of less than ±0.27° C. where the measured physiological data are used to derive a decision tree to predict Tc of the user.

20. The method of claim 17, wherein the step of measuring the physiological data of the user is repeated according to a pre-defined time interval so that Tc of the user can be monitored continuously.
